(19) United States
(12) Patent Application Publication
(10) Pub. No.: US 2008/0097566 A1
(43) Pub. Date: Apr. 24, 2008

(54) FOCUSED SEGMENTED ELECTRODE

(76) Inventor: Olivier Colliou, Los Gatos, CA (US)

Correspondence Address: BOZICEVIC, FIELD & FRANCIS LLP;(PROTEUS BIOMEDICAL, INC), 1900 UNIVERSITY AVENUE, SUITE 200, EAST PALO ALTO, CA, 94303, US

(21) Appl. No.: 11/777,981

(22) Filed: Jul. 13, 2007

Related U.S. Application Data
(60) Provisional application No. 60/807,289, filed on Jul. 13, 2006.

(51) Int. Cl. A61N 1/00

(52) U.S. Cl. 607/122; 607/115

(57) ABSTRACT

The present invention provides significantly improved electrode structures, including segmented electrode structures, which are able to deliver highly focused energy to tissue when implanted into a patient. Embodiments of the invention include focused segmented electrodes. Also provided are leads that include the focused segmented electrodes, implantable pulse generators that include the leads, as well as systems and kits having components thereof, and methods of making and using the subject devices.

### CROSS-REFERENCE TO RELATED APPLICATIONS

Pursuant to 35 U.S.C. § 119 (e), this application claims priority to U.S. Provisional Application Ser. No. 60/807,289 filed Jul. 13, 2006; the disclosure of which priority application is herein incorporated by reference.

### INTRODUCTION

1. Field of the Invention

The present invention relates generally to implantable medical devices.

2. Background

Pacemakers and other implantable medical devices find wide-spread use in today's health care system. A typical pacemaker includes stimulating electrodes that are placed in contact with heart muscle, detection electrodes placed to detect movement of the heart muscle, and control circuitry for operating the stimulating electrodes based on signals received from the detection electrodes. Thus, the pacemaker can detect abnormal (e.g., irregular) movement and deliver electrical pulses to the heart to restore normal movement.

Pacing leads implanted in vessels in the body are, for many applications, flexible cylindrical devices. They are cylindrical due to three main reasons: most anatomical conduits are cylindrical, medical sealing and access devices seal on cylindrical shapes and cylindrical leads have uniform bending moments of inertia around the long axis of the device. The cylindrical nature of the device necessitates the cylindrical design of pacing electrodes on the body of the device.

Due to the tortuous nature of the vessels in the body, following implantation the rotational orientation of one electrode can not be predetermined in many currently employed devices. As such, many currently employed lead devices employ cylindrical electrode designs that are conductive to tissue around the entirety of the diameter of the lead. This insures that some portion of the cylindrical electrode contacts excitable tissue when they are implanted. Despite the multiple devices in which cylindrical continuous ring electrodes are employed, there are disadvantages to such structures, including but not limited to: undesirable excitation of non-target tissue, e.g., which can cause unwanted side effects, increased power use, etc.

An innovative way to address this problem is to employ segmented electrode structure, in which the circular band electrode is replaced by an electrode structure made up of two or more individually activatable and electrically isolated electrode structures that are configured in a discontinuous band. Such segmented electrode structures are disclosed in published PCT application Publication Nos. WO 2006/069322 and WO2006/029090; the disclosures of which are herein incorporated by reference.

While providing significant improvements in functionality, there is continued interest in the development of improved segmented electrode structures which are more structurally robust.

### SUMMARY

The present invention provides significantly improved electrode structures, including segmented electrode structures, which are able to deliver highly focused energy to tissue when implanted into a patient. Embodiments of the invention include focused segmented electrodes. Also provided are leads that include the focused segmented electrodes, implantable pulse generators that include the leads, as well as systems and kits having components thereof, and methods of making and using the subject devices.

### BRIEF DESCRIPTION OF THE FIGURES

FIG. 1 provides a depiction of a focused segmented electrode that may be employed in focused stimulation, in accordance with an embodiment of the invention, where the focused segmented electrode comprises a series of juxtaposed strip electrodes;

FIG. 2 provides a depiction of a focused segmented electrode that may be employed in focused stimulation, in accordance with an embodiment of the invention, where the focused segmented electrode comprises a single central electrode and single outer electrode that circumscribes the central electrode;

FIG. 3 provides a depiction of a focused segmented electrode that may be employed in focused stimulation, in accordance with an embodiment of the invention, where the focused segmented electrode comprises four sets of juxtaposed strip electrodes arranged circumferentially about a lead;

FIG. 4 provides a depiction of a focused segmented electrode that may be employed in focused stimulation, in accordance with an embodiment of the invention, where the focused segmented electrode comprises a single disc shaped central electrode and single outer ring-shaped electrode that circumscribes the central electrode (i.e., an electrode having a “bulls-eye” configuration);

FIG. 5A provides a depiction of a focused segmented electrode that may be employed in focused stimulation, in accordance with an embodiment of the invention, where the focused segmented electrode comprises a single disc shaped central electrode and single outer electrode that circumscribes the central electrode (i.e., an electrode having a “bulls-eye” configuration); while FIG. 5B provides a cross-sectional view of the same electrode along line A-A;

FIG. 6 provides a depiction of a focused segmented electrode that may be employed in focused stimulation, in accordance with an embodiment of the invention, where the focused segmented electrode comprises a single square shaped central electrode and 9 different outer electrodes that collectively circumscribe the central electrode;

FIG. 7A provides a depiction of a side view of a focused voltage gradient produced by a focused segmented electrode according to an embodiment of the invention; while FIG. 7B provides a depiction of an end on view of the same focused segmented electrode; and

FIG. 8 provides a depiction of a cardiac resynchronization therapy system that includes one or more hermetically sealed integrated circuits coupled to lead electrodes according to an embodiment of the invention.

### DETAILED DESCRIPTION

As summarized above, the present invention provides significantly improved satellite electrode structures, including segmented electrode structures, which are which are able to deliver highly focused energy to tissue when implanted into a patient. Embodiments of the invention include focused segmented electrodes, where the electrodes may be present on a flexible medical carrier, e.g., vascular lead. Also provided are leads that include focused segmented electrodes, implantable pulse generators that include the leads, as well as systems and kits having components thereof, and methods of making and using the subject devices.

In further describing aspects of the invention in greater detail, embodiments of focused segmented electrode, as well as medical carriers and medical devices that include the same, is provided. In addition, a further description of kits and systems of the invention, and methods of using various aspects of the invention, is provided.

Focused Segmented Electrodes

Embodiments of the invention include focused segmented electrode assemblies, such as electrode satellite structures, where the structures include focused segmented electrode. In further embodiments, the satellite structures may include control circuitry, e.g., in the form of an IC (e.g., an IC inside of the support), such that the satellite structure is addressable. In certain embodiments, the structure includes two or more electrode elements, such as three or more electrode elements, including four or more electrode elements.

The assemblies are configured to deliver focused energy to a tissue location during use. As such, highly focused electrical currents are produced in tissue upon activation of the focused electrode assemblies, as reviewed below in greater detail.

In certain embodiments, the supports are configured for use in segmented electrode structures. By segmented electrode structure is meant an electrode structure that includes two or more, e.g., three or more, including four or more, disparate electrode elements. Embodiments of segmented electrode structures are disclosed in Application Serial Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; and Ser. No. 11/734,617 titled “High Phrenic, Low Pacing Capture Threshold Pacing Devices and Methods” filed on Apr. 12, 2007; the disclosures of the various segmented electrode structures of these applications being herein incorporated by reference.

In certain embodiments, the focused segmented electrodes are “addressable” electrode structures. Addressable electrode structures include structures having one or more electrode elements directly coupled to control circuitry, e.g., present on an integrated circuit (IC). Addressable electrodes include satellite structures that include one more electrode elements directly coupled to an IC and configured to be placed along a lead. Examples of addressable electrode structures that include an IC are disclosed in application Ser. No. 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; and Ser. No. 11/734,617 titled “High Phrenic, Low Pacing Capture Threshold Pacing Devices and Methods” filed Apr. 12, 2007; the disclosures of the various addressable electrode structures of these applications being herein incorporated by reference. In these embodiments where an IC is present, the segmented electrode structure may include IC holding elements that immobilize an IC relative to the other elements of the structure.

In certain embodiments, the area of the anode is greater than the area of the cathode, e.g., by a factor of about 3:1 or more, such as by a factor of about 10:1 or more, including by factor of about 15:1 or more. In certain embodiments, the anode element(s) may surround or circumscribe the cathode elements. In yet other embodiments, the anode elements may be inter-digitated with the cathode elements.

The segmented electrode structures may vary considerably, so long as the different electrode elements are sufficiently proximal to each other to generate the desired electric stimulation. Distances between the electrode structures may vary, where in certain embodiments, the distances are about 1000 μm or less, such as about 500 μm or less, and in certain embodiments range from about 5 μm to about 1000 μm, such as from about 50 μm to about 500 μm and including from about 100 to about 300 μm, e.g., about 200 μm.

Where the segmented electrode structure is present on a lead or analogous carrier, the electrode structure may be conductively coupled to an elongated conductive member, e.g., to provide for communication with a remote structure, such as a remote controller, e.g., which may be present in a structure which is known in the art as a “can.” As such, in certain embodiments, the segmented electrode structures are electrically coupled to at least one elongated conductor, which elongated conductor may or may not be present in a lead, and may or may not in turn be electrically coupled to a control unit, e.g., that is present in a pacemaker can. In such embodiments, the combination of segmented electrode structure and elongated conductor may be referred to as a lead assembly.

In certain embodiments, each electrode element of the segmented structure may be coupled to its own conductive member or members, such that each electrode element is coupled to its own wire. In these embodiments the structure or carrier, e.g., lead, on which the structure is present may be torqueable, such that it can be turned during and upon placement of the lead so that upon activation, the electrode elements produce stimulation in the desired, focused direction.

In yet other embodiments, the electrode elements of the structure are present on a multiplex lead, such that two or more disparate electrode structures are coupled to the same lead or leads. A variety of multiplex lead formats are known in the art and may readily be adapted for use in the present devices. See e.g., U.S. Pat. Nos. 5,593,430; 5,999,848; 6,418,348; 6,421,567 and 6,473,653; the disclosures of which are herein incorporated by reference. Of particular interest are multiplex leads as disclosed in published U.S. Patent application no. 2004-0193021; the disclosure of which is herein incorporated by reference.

Of interest are structures that include an integrated circuit (IC) electrically coupled (so as to provide an electrical connection) to two or more electrode elements. The term “integrated circuit” (IC) is used herein to refer to a tiny complex of electronic components and their connections that is produced in or on a small slice of material, i.e., chip, such as a silicon chip. In certain embodiments, the IC is a multiplexing circuit, e.g., as disclosed in PCT Application No. PCT/US2005/031559 titled “Methods and Apparatus for Tissue Activation and Monitoring” and filed on Sep. 1, 2005; the disclosure of which is herein incorporated by reference. In the segmented electrode structures, the number of electrodes that is electrically coupled to the IC may vary, where in certain embodiments the number of 2 or more, e.g., 3 or more, 4 or more, etc., and in certain embodiments ranged from 2 to about 20, such as from about 3 to about 8, e.g., from about 4 to about 6. While being electrically coupled to the IC, the different electrodes of the structures are electrically isolated from each other, such that current cannot flow directly from one electrode to the other. In these embodiments, the lead need not be torqueable, since the desired focused stimulation can be achieved through selective activation of electrodes.

As summarized above, the invention provides implantable medical devices that include the electrode structures as described above. By implantable medical device is meant a device that is configured to be positioned on or in a living body, where in certain embodiments the implantable medical device is configured to be implanted in a living body. Embodiments of the implantable devices are configured to maintain functionality when present in a physiological environment, including a high salt, high humidity environment found inside of a body, for 2 or more days, such as about 1 week or longer, about 4 weeks or longer, about 6 months or longer, about 1 year or longer, e.g., about 5 years or longer. In certain embodiments, the implantable devices are configured to maintain functionality when implanted at a physiological site for a period ranging from about 1 to about 80 years or longer, such as from about 5 to about 70 years or longer, and including for a period ranging from about 10 to about 50 years or longer. The dimensions of the implantable medical devices of the invention may vary. However, because the implantable medical devices are implantable, the dimensions of certain embodiments of the devices are not so big such that the device cannot be positioned in an adult human.

Embodiments of the invention also include medical carriers that include one or more focused segmented electrode assemblies, e.g., as described above. Carriers of interest include, but are not limited to, vascular lead structures, where such structures are generally dimensioned to be implantable and are fabricated from a physiologically compatible material. With respect to vascular leads, a variety of different vascular lead configurations may be employed, where the vascular lead in certain embodiments is an elongated tubular, e.g., cylindrical, structure having a proximal and distal end. The proximal end may include a connector element, e.g., an IS-1 connector, for connecting to a control unit, e.g., present in a “can” or analogous device. The lead may include one or more lumens, e.g., for use with a guidewire, for housing one or more conductive elements, e.g., wires, etc. The distal end may include a variety of different features as desired, e.g., a securing means, etc.

In certain embodiments of the subject systems, one or more sets of electrode assemblies or satellites as described above are electrically coupled to at least one elongated conductive member, e.g., an elongated conductive member present in a lead, such as a cardiovascular lead. For example, two or more assemblies are coupled to a common at least one electrical conductor, i.e., to the same at least one electrical conductor. In certain embodiments, the elongated conductive member is part of a multiplex lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or less wires, such as only 2 wires or only 1 wire. Multiplex lead structures of interest include those described in application Ser. No. 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; and Ser. No. 11/734,617 titled “High Phrenic, Low Pacing Capture Threshold Pacing Devices and Methods” filed Apr. 12, 2007; the disclosures of the various multiplex lead structures of these applications being herein incorporated by reference. In some embodiments of the invention, the devices and systems may include onboard logic circuitry or a processor, e.g., present in a central control unit, such as a pacemaker can. In these embodiments, the central control unit may be electrically coupled to the lead by a connector, such as a proximal end IS-1 connection.

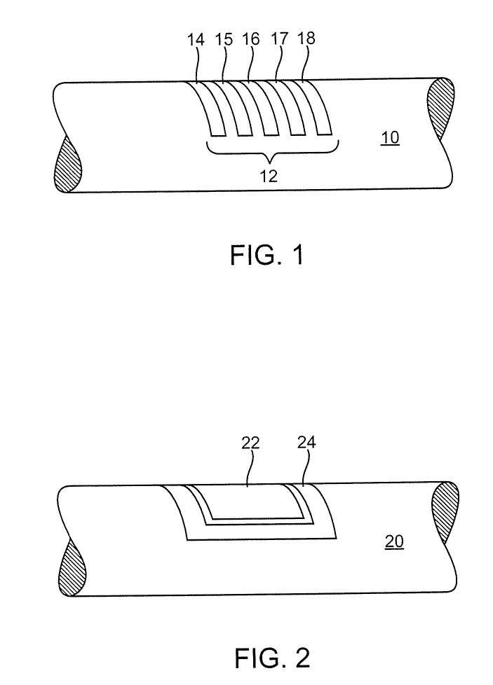

FIG. 1 provides a depiction of a focused segmented electrode that may be employed in focused stimulation, in accordance with an embodiment of the invention, where the focused segmented electrode comprises a series of juxtaposed strip electrodes. In FIG. 1, the segmented electrode structure 12 on lead body 10 is a series of five juxtaposed strip electrodes, 13, 14, 15, 16 and 18, which are positioned on only a portion or region of a lead body 10, such that they do not circumscribe the lead body. Electrodes 14, 16 and 18 are operated as anodes and electrodes 13 and 15 are operated as cathodes. By activating the strip electrode elements of the structure to provide for an alternating anode-cathode configuration, highly focused bipolar stimulation is produced. The structure depicted in FIG. 1 can be viewed as an inter-digitated structure.

FIG. 2 provides a depiction of a focused segmented electrode that may be employed in focused stimulation, in accordance with an embodiment of the invention, where the focused segmented electrode comprises a single central electrode and single outer electrode that circumscribes the central electrode. In FIG. 2, the segmented electrode structure has a “bull's-eye” configuration, with a single central cathode 22 and surrounding annular anode 24, present on lead body 20. Through appropriate activation, the current is focused in the gap between anode and cathode and therefore focused into the surrounding tissue. The depth to which the current penetrates the surrounding tissue can be controlled by choosing the appropriate anode-to-cathode spacing.

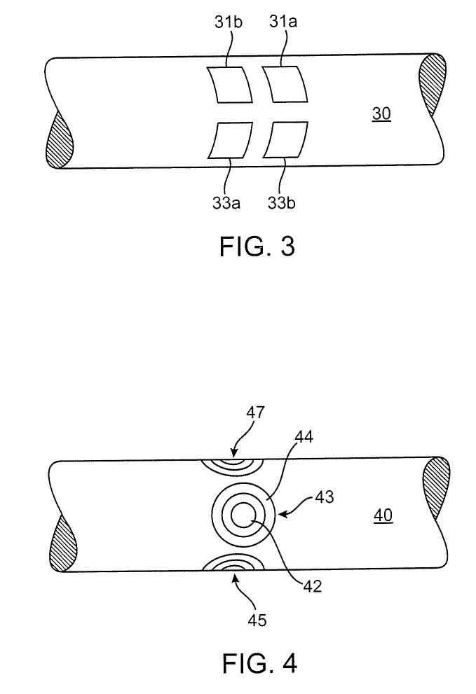

FIG. 3 provides a depiction of a focused segmented electrode that may be employed in focused stimulation, in accordance with an embodiment of the invention, where the focused segmented electrode comprises four sets (only two of which sets are shown in the figure) of juxtaposed strip electrodes arranged circumferentially about a lead 30. FIG. 3 provides another view of a strip electrode configuration, where separated rectangular electrode elements 31a, 31b (making up set 1), and 33a and 33b (making up set 2), are positioned next to each other on the surface of lead 30 and circumscribe a lead body with two more sets not shown.

FIG. 4 provides a depiction of a focused segmented electrode that may be employed in focused stimulation, in accordance with an embodiment of the invention, where the focused segmented electrode comprises a single disc shaped central electrode and single outer ring-shaped electrode that circumscribes the central electrode (i.e., an electrode having a “bulls-eye” configuration). As such, FIG. 4 provides yet another view of a “bull's eye” electrode configuration, wherein a central electrode element 42, e.g., a cathode, is surrounded by a second annular electrode element 44, e.g., an annular anode. In this “bull's eye” configuration, multiple “bull's eye” electrodes 43, 45 and 47 circumscribe the lead body 40 allowing the selectability of the best “bull's eye” which is oriented towards the cardiac muscle. This in turn provides a highly focused electrical field into the cardiac muscle.

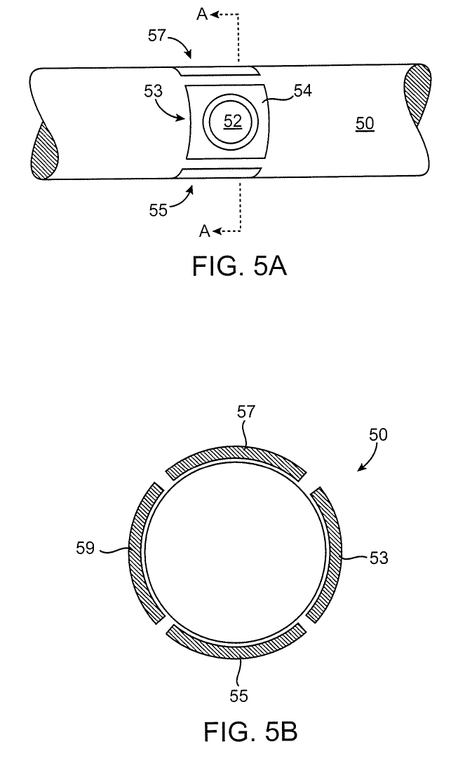

FIG. 5A provides a depiction of a focused segmented electrode that may be employed in focused stimulation, in accordance with an embodiment of the invention, where the focused segmented electrode comprises a single disc shaped central electrode and single outer electrode that circumscribes the central electrode (i.e., an electrode having a “bulls-eye” configuration); while FIG. 5B provides a cross-sectional view of the same electrode along line A-A of FIG. 5A. As such, FIG. 5A provides a view of a yet another embodiment in which a first central electrode element 52 is surrounded by a second electrode element 54. Second or outer electrode element has a square outer shape and a circular inner shape. Note that there are many more electrode shapes that are possible, such that the shapes depicted in these figures are merely illustrative and not limiting. Electrodes 52 and 54 together make up focused segmented electrode 53. Focused segmented electrode 53, along with focused segmented electrodes 55, 57 and 59, are arranged circumferentially about lead body 50, as shown in FIG. 5A and also in FIG. 5B.

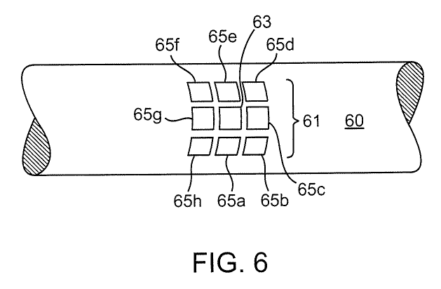

FIG. 6 provides a depiction of a focused segmented electrode that may be employed in focused stimulation, in accordance with an embodiment of the invention, where the focused segmented electrode 61 comprises a single square shaped central electrode 63 and 9 different outer electrodes (i.e., 65a, 65b, 65c, 65d, 65e, 65f, 65g, and 65h, that collectively circumscribe the central electrode. This configuration provides an even higher selectability of orientations for the “bull's eye” focused stimulation by allowing the middle row of electrodes circumscribing the lead to act as cathodes or anodes.

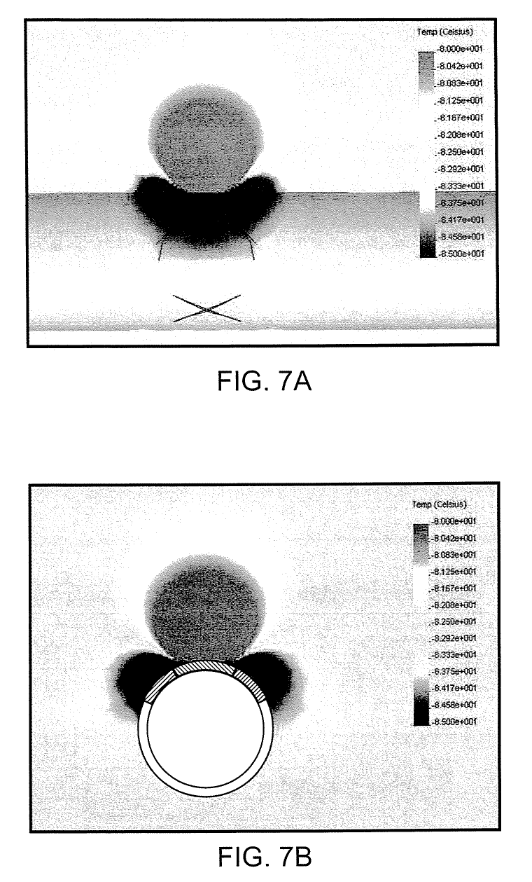

FIG. 7A provides a depiction of a side view of a focused voltage gradient produced by a focused segmented electrode having a “bulls-eye” configuration according to an embodiment of the invention; while FIG. 7B provides a depiction of an end on view of the same focused segmented electrode; and

The leads may further include a variety of different effector element, which elements may employ the satellites or structures distinct from the satellites. The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. patent application Ser. No. 10/734,490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

Implantable Pulse Generators

Embodiments of the invention further include implantable pulse generators. Implantable pulse generators may include: a housing which includes a power source and an electrical stimulus control element; one or more vascular leads as described above, e.g., 2 or more vascular leads, where each lead is coupled to the control element in the housing via a suitable connector, e.g., an IS-1 connector. In certain embodiments, the implantable pulse generators are ones that are employed for cardiovascular applications, e.g., pacing applications, cardiac resynchronization therapy applications, etc. As such, in certain embodiments the control element is configured to operate the pulse generator in a manner so that it operates as a pacemaker, e.g., by having an appropriate control algorithm recorded onto a computer readable medium of a processor of the control element. In certain embodiments the control element is configured to operate the pulse generator in a manner so that it operates as a cardiac resynchronization therapy device, e.g., by having an appropriate control algorithm recorded onto a computer readable medium of a processor of the control element.

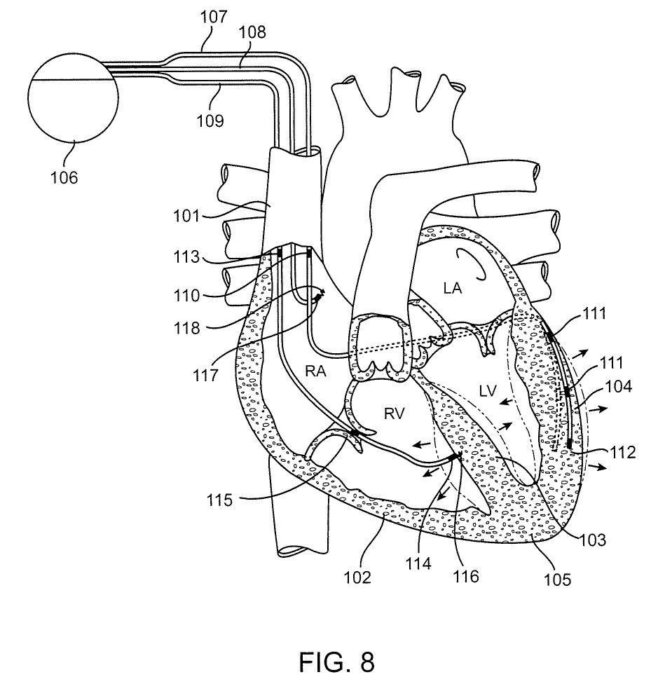

FIG. 8 provides a depiction of a cardiac resynchronization therapy system that includes one or more hermetically sealed integrated circuits coupled to lead electrodes according to an embodiment of the invention. An implantable pulse generator according to an embodiment of the invention is depicted in FIG. 8, which provides a cross-sectional view of the heart with of an embodiment of a cardiac resynchronization therapy (CRT) system. The system includes a pacemaker can 106 that includes a control element (e.g., processor) and a power source, a right ventricle electrode lead 109, a right atrium electrode lead 108, and a left ventricle cardiac vein lead 107. Also shown are the right ventricle lateral wall 102, interventricular septal wall 103, apex of the heart 105, and a cardiac vein on the left ventricle lateral wall 104.

The left ventricle electrode lead 107 is comprised of a lead body and one or more satellite electrode assemblies 110,111, and 112. Each of the electrodes assemblies is a satellite as described above and includes a focused segmented electrode assembly, e.g., as shown in any of FIGS. 1 to 7B. Having multiple distal electrode assemblies allows a choice of optimal electrode location for CRT. In a representative embodiment, electrode lead 107 is constructed with the standard materials for a cardiac lead such as silicone or polyurethane for the lead body, and MP35N for the coiled or stranded conductors connected to Pt—Ir (90% platinum, 10% iridium) electrode assemblies 110,111 and 112. Alternatively, these device components can be connected by a multiplex system (e.g., as described in published United States Patent Application publication nos.: 20040254483 titled “Methods and systems for measuring cardiac parameters”; 20040220637 titled “Method and apparatus for enhancing cardiac pacing”; 20040215049 titled “Method and system for remote hemodynamic monitoring”; and 20040193021 titled “Method and system for monitoring and treating hemodynamic parameters; the disclosures of which are herein incorporated by reference), to the proximal end of electrode lead 107. The proximal end of electrode lead 107 connects to a pacemaker 106, e.g., via an IS-1 connector.

The electrode lead 107 is placed in the heart using standard cardiac lead placement devices which include introducers, guide catheters, guidewires, and/or stylets. Briefly, an introducer is placed into the clavicle vein. A guide catheter is placed through the introducer and used to locate the coronary sinus in the right atrium. A guidewire is then used to locate a left ventricle cardiac vein. The electrode lead 107 is slid over the guidewire into the left ventricle cardiac vein 104 and tested until an optimal location for CRT is found. Once implanted a multi-electrode lead 107 still allows for continuous readjustments of the optimal electrode location.

The electrode lead 109 is placed in the right ventricle of the heart with an active fixation helix at the end 116 which is embedded into the cardiac septum. In this view, the electrode lead 109 is provided with one or multiple electrodes 113,114,115.

Electrode lead 109 is placed in the heart in a procedure similar to the typical placement procedures for cardiac right ventricle leads. Electrode lead 109 is placed in the heart using the standard cardiac lead devices which include introducers, guide catheters, guidewires, and/or stylets. Electrode lead 109 is inserted into the clavicle vein, through the superior vena cava, through the right atrium and down into the right ventricle. Electrode lead 109 is positioned under fluoroscopy into the location the clinician has determined is clinically optimal and logistically practical for fixating the electrode lead 109. Under fluoroscopy, the active fixation helix 116 is advanced and screwed into the cardiac tissue to secure electrode lead 109 onto the septum. The electrode lead 108 is placed in the right atrium using an active fixation helix 118. The distal tip electrode 118 is used to both provide pacing and motion sensing of the right atrium.

Summarizing aspects of the above description, in using the implantable pulse generators of the invention, such methods include implanting an implantable pulse generator e.g., as described above, into a subject; and the implanted pulse generator, e.g., to pace the heart of the subject, to perform cardiac resynchronization therapy in the subject, etc. The description of the present invention is provided herein in certain instances with reference to a subject or patient. As used herein, the terms “subject” and “patient” refer to a living entity such as an animal. In certain embodiments, the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects, e.g., patients, are humans.

During operation, use of the implantable pulse generator may include activating at least one of the electrodes of the pulse generator to deliver electrical energy to the subject, where the activation may be selective, such as where the method includes first determining which of the electrodes of the pulse generator to activate and then activating the electrode. Methods of using an IPG, e.g., for pacing and CRT, are disclosed in Application Serial Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; and Ser. No. 11/734,617 titled “High Phrenic, Low Capture Threshold Pacing Devices and Methods,” filed Apr. 12, 2006; the disclosures of the various methods of operation of these applications being herein incorporated by reference and applicable for use of the present devices.

The devices and systems of the invention may find use in, methods of highly specific tissue stimulation, e.g., highly specific cardiac tissue stimulation. As such, the invention includes methods of focused cardiac tissue stimulation. By focused cardiac tissue stimulation is meant that electrical stimulation is generated from an electrode structure in an asymmetric directional manner from the electrode structure, such that the electrode structure does not provide symmetrical electrical stimulation to the same extent into all tissue surrounding the electrode structure. In certain embodiments, focused stimulation arises from a bipolar electrode structure, e.g., from an electrode structure having at least one anode and at least one cathode which are sufficient proximal to each other that, upon application of a suitable stimulatory current, an electrical stimulation is produced in the tissue that is contacted by the anode and the cathode. As the stimulations of the subject methods are selective, they have a high selectivity ratio, where selectivity ratio is determined by the formula:

Selectivity=unwanted nerve capture voltage/desired tissue capture voltage. In certain embodiments, the selectivity ratio of the subject methods is about 5 or higher, such as about 10 or higher and including about 15 or higher, e.g., 20 or higher.

Where the methods are methods of selective cardiac tissue stimulation with respect to the phrenic nerve, selectivity as determined using the following formula:
Selectivity=phrenic nerve capture voltage/cardiac capture voltage
is about 5 or higher, such as about 10 or higher and including about 15 or higher, e.g., 20 or higher.

The selective stimulation feature of the subject methods also provides for embodiments of tissue stimulation in which the amount of voltage needed for effective capture is less than that employed in methods where tissue is not selectively stimulated. For example, in certain cardiac tissue stimulation methods, effective cardiac capture is achieved with voltages of about 10 volts or less e.g., about 5 volts or less, such as about 1.5 volts or less, including about 0.50 volts or less, such as about 0.25 volts or less.

Where the tissue that is stimulated in the subject methods is cardiac tissue, embodiments of the methods of cardiac tissue stimulation may be characterized as high phrenic nerve capture threshold, low cardiac tissue capture threshold methods. In these embodiments, cardiac tissue is stimulated in a manner such that the capture threshold for the phrenic nerve is significantly higher than the capture threshold for the cardiac tissue, e.g., about 5 times or more higher, such about 10 times or more higher and including about 20 times more or higher. In certain embodiments, the capture of the phrenic nerve only occurs with activation energies of about 3 to about 18 volts or higher, such as about 10 to about 17 volts or higher, including about 15 volts or higher.

Where desired, the methods may include a step of obtaining phrenic nerve capture data and employing this data in the selective tissue stimulation. For example, a sensor can be employed to detect phrenic nerve capture, and the resultant data employed to set or more modify the cardiac stimulation parameters of focused cardiac stimulation. The sensor may be present in the same lead or a different lead from the cardiac stimulation lead. Any convenient sensor may be employed. The sensor could be an electrical sensor if it is on the diaphragm or near the phrenic nerve or it could be a motion sensor or a mechanical motion sensor on the lead. Examples of suitable sensors include pressure sensors, strain gauges, accelerometers, acoustic sensors, where the sensors can be orientated anywhere along the lead or independently on another lead placed on the diaphragm.

In certain embodiments, feedback regarding phrenic nerve capture or lack thereof is provided so that if one is automatically repositioning electrodes the box can have a feedback mechanism and the circuit can make sure that it does not choose an inappropriate electrode that would cause phrenic stimulation. In addition, during the initial programming of the device it could provide feedback that would be sub-threshold or tactile threshold for the clinician when he is observing the patient or possibly also for the patient.

In other embodiments, data regarding phrenic nerve capture, e.g., from distinct devices associated with the diaphragm, such as a diaphragm lead, can be employed. Any convenient method of communicating the data from the diaphragm specific lead to the controller of the pacing lead may be employed, such as an RF or other suitable communication protocol.

As such, the phrenic nerve capture device could be inside the cardiac stimulation lead or associated with a deminimus ASIC chip or it could be a separate packaged assembly inside the lead and not exposed.

One can evaluate for a correlation between pacing pulses and EMG signals around diaphragm or phrenic nerve signals.

Another suitable protocol for testing for phrenic nerve capture is to use non-cardiac tissue pace inducing pulses, such as pulses at a higher frequency, at a different rate that the pace rate, e.g., slower than a cardiac pacing rate, or a different series of wave forms to test for phrenic capture independently of pacing. Alternatively, test pulses during the heart's refractory period may be generated. Such protocols may employ an external communicating device that could be positioned on the outside of the patient that would detect the higher frequency motions and then relay that to either the ICD in the person's chest or the computing device in the person's chest or the computer when this is going through programming. This device could also be attached so that if the pacing parameters are changed during an exercise or a stress test this could provide feedback during an exercise or stress test assuming the frequency of the vibrations would be detectable when it is overlaid on top of any kind of motion and this could be used during the night to monitor a patient over a period of days with an external device that would provide this detection and this device could be internally implanted. This device could be either attached through a lead or have an antenna and have radio frequency communication that would detect phrenic capture. This device would evaluate at the data set for the data from the different sensors so the data change of interest would be the data change that happened concurrently with pacing pulses. That would include both pressure changes and motion changes and, where desired, electrical pacing on a diaphragm on the surface of the diaphragm or near the diaphragm. So this device could also be an adhesively applied patch that would be applied to the patient over a period of from 1 hour to 24 or 48 hours. The device need not be continuously powered, but may be powered only during times when change is occurring. So if the ICD thinks it is about ready to try a different pacing location then one could turn on the sensor just to get feedback about phrenic nerve capture. Where desired, this sensor would be running for a period of time to catch several breath cycles do to the erratic nature of the capture of the phrenic nerve.

The above described methods of detecting phrenic nerve capture and employing the capture data in pacing are merely representative. The obtained phrenic nerve capture data may be employed in a number of different ways, such as in the initial determination of a pacing protocol (such as which electrodes of a segmented electrode structure to activate, the voltage to employ, etc.), in the modification of an existing pacing protocol, etc. In certain embodiments, the feedback may be open loop, such that phrenic nerve capture data is evaluated by a health care practitioner. The data may be provided in terms of a safety factor, e.g., ratio of heart capture threshold to phrenic nerve capture threshold during implant. As desired the health care practitioner may then set pacing parameters based on the phrenic nerve capture data. In yet other embodiments, the feedback is closed loop, such that a pacing protocol is automatically adjusted in response to the obtained phrenic nerve capture date, e.g., by a processor in an ICD or even by a processor in a chip that is part of a segmented electrode structure.

In practicing the subject methods, any convenient electrical stimulation device that can provide for the selective tissue, e.g., cardiac tissue, stimulation may be employed. One type of device that may be employed in the subject methods is a segmented electrode device, i.e., a device that includes a segmented electrode structure. As summarized above, a segmented electrode structure is an electrode structure made up of two or more distinct electrode elements positioned proximal to each other, e.g., on a support such as a lead, where the electrode elements can be activated in a manner sufficient to provide for selective tissue stimulation, e.g., as described above. The segmented electrode structures may be configured to produce bipolar electrical stimulation, in which one of the electrode elements of the structure acts as the anode and the other electrode element(s) acts as the cathode, such that an electrical field is generated between the electrode elements which provides focused stimulation to the tissue in contact with the segmented electrode structure.

In certain segmented electrode embodiments, the methods include “pacing” between electrode elements of the same band, i.e., between two or more of the electrode components of the same segmented electrode structure. As such, these embodiments are distinguished from non-segmented electrode applications in which pacing may occur between two different bands on a lead, since the embodiments of the subject invention may be characterized as intraband pacing embodiments, as opposed to interband pacing embodiments.

Systems

Also provided are systems that include one more devices as described above. The systems of the invention may be viewed as systems for communicating information within the body of subject, e.g., human, where the systems include both a first implantable medical device, such as an IPG device described above, that includes a transceiver configured to transmit and/or receive a signal; and a second device comprising a transceiver configured to transmit and/or receive a signal. The second device may be a device that is inside the body, on a surface of the body or separate from the body during use.

Also provided are methods of using the systems of the invention. The methods of the invention generally include: providing a system of the invention, e.g., as described above, that includes first and second medical devices, one of which may be implantable; and transmitting a signal between the first and second devices. In certain embodiments, the transmitting step includes sending a signal from the first to said second device. In certain embodiments, the transmitting step includes sending a signal from the second device to said first device. The signal may transmitted in any convenient frequency, where in certain embodiments the frequency ranges from about 400 to about 405 MHz. The nature of the signal may vary greatly, and may include one or more data obtained from the patient, data obtained from the implanted device on device function, control information for the implanted device, power, etc.

Use of the systems may include visualization of data obtained with the devices. Some of the present inventors have developed a variety of display and software tools to coordinate multiple sources of sensor information which will be gathered by use of the inventive systems. Examples of these can be seen in international PCT application serial no. PCT/US2006/012246; the disclosure of which application, as well as the priority applications thereof are incorporated in their entirety by reference herein.

Kits

Also provided are kits that include the subject electrode structures, as part of one or more components of an implantable device or system, such as an implantable pulse generator, e.g., as reviewed above. In certain embodiments, the kits further include at least a control unit, e.g., in the form of a pacemaker can. In certain of these embodiments, the structure and control unit may be electrically coupled by an elongated conductive member. In certain embodiments, the electrode structure may be present in a lead, such as a cardiovascular lead.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An implantable elongated flexible structure comprising a proximal end and a distal end, wherein said structure further comprises a focused segmented electrode, wherein said focused segmented electrode comprises two or more electrodes conductively coupled to an individually addressable processor, wherein said electrodes are configured to deliver tissue-focused stimulation.

2. The implantable elongated flexible structure according to claim 1, wherein said focused segmented electrode comprises electrodes positioned on only one side of said lead.

3. The implantable elongated flexible structure according to claim 2, wherein said focused segmented electrode comprises a series of juxtaposed strip electrodes.

4. The implantable elongated flexible structure according to claim 1, wherein said focused segmented electrode comprises a central electrode and one or outer electrodes peripherally arranged about said central electrode.

5. The implantable elongated flexible structure according to claim 4, wherein said focused segmented electrode comprises a single outer electrode that circumscribes said central electrode.

6. The implantable elongated flexible structure according to claim 4, wherein said focused segmented electrode comprises two or more outer electrodes that collectively circumscribe said central electrode.

7. The implantable elongated flexible structure according to claim 1, wherein said structure is a vascular lead.

8. The implantable elongated flexible structure according to claim 7, wherein said vascular lead comprises 2 or more individually addressable focused segmented electrodes.

9. The implantable elongated flexible structure according to claim 8, wherein said vascular lead is a multiplex lead having 3 or less wires.

10. The elongated flexible structure according to claim 9, wherein said vascular lead includes only 2 wires.

11. The elongated flexible structure according to claim 10, wherein said vascular lead includes only 1 wire.

12. The elongated flexible structure according to claim 11, wherein said vascular lead includes an IS-1 connector at said proximal end.

13. An implantable pulse generator comprising:
(a) a housing comprising a power source and an electrical stimulus control element; and
(b) a vascular lead comprising a focused segmented electrode, wherein said focused segmented electrode comprises two or more electrodes conductively coupled to an individually addressable processor, wherein said electrodes are configured to deliver tissue-focused stimulation.

14. The implantable pulse generator according to claim 13, wherein said control element is configured to operate said implantable pulse generator as a pacemaker.

15. The implantable pulse generator according to claim 13, wherein said control element is configured to operate said implantable pulse generator in a manner sufficient to achieve cardiac resynchronization.

16. A method comprising:
(a) implanting into a patient an implantable pulse generator comprising:
(i) a housing comprising a power source and an electrical stimulus control element; and
(ii) a vascular lead comprising a focused segmented electrode, wherein said focused segmented electrode comprises two or more electrodes conductively coupled to an individually addressable processor, wherein said electrodes are configured to deliver tissue-focused stimulation; and
(b) delivering electrical stimulation to tissue of said patient from said focused segmented electrode.

17. The method according to claim 16, wherein said tissue is cardiac tissue.

18. The method according to claim 17, wherein said method is a method of cardiac pacing.

19. The method according to claim 17, wherein said method is a method of cardiac resynchronization therapy.

20. A kit comprising:
(a) a housing comprising a power source and an electrical stimulus control element; and
(b) a vascular lead comprising a focused segmented electrode, wherein said focused segmented electrode comprises two or more electrodes conductively coupled to an individually addressable processor, wherein said electrodes are configured to deliver tissue-focused stimulation.
